(19) United States
(12) Patent Application Publication
(10) Pub. No.: US 2022/0054707 A1
(43) Pub. Date: Feb. 24, 2022

(54) BIOLOGICAL MATERIAL WITH COMPOSITE EXTRACELLULAR MATRIX COMPONENTS

(71) Applicants: Shanghai Zhuoruan Medical Technologies Co., Ltd, Shanghai (CN); Zhuoruan Medical Technologies (Suzhou) Co., Ltd, Taicang (CN)

(72) Inventors: Jian ZHANG, Taicang (CN); Yuanyuan WANG, Taicang (CN)

(73) Assignees: Shanghai Zhuoruan Medical Technologies Co., Ltd, Shanghai (CN); Zhuoruan Medical Technologies (Suzhou) Co., Ltd, Taicang (CN)

(21) Appl. No.: 17/518,124

(22) Filed: Nov. 3, 2021

Related U.S. Application Data
(63) Continuation-in-part of application No. 16/095,633, filed on Oct. 22, 2018, now abandoned.

(30) Foreign Application Priority Data
Apr. 25, 2016 (CN) 201610261472.1
Sep. 30, 2021 (CN) 202111160236.8
Sep. 30, 2021 (CN) 202111163004.8
Oct. 13, 2021 (CN) 202111192492.5

(51) Int. Cl. A61L 27/36

(52) U.S. Cl. CPC A61L 27/3633; A61L 27/3629; A61L 2430/40; A61L 2430/34; A61L 27/3683

(57) ABSTRACT

A biological material with composite extracellular matrix component, in which decellularized small intestinal submucosa (SIS) is treated as the interlayer and decellularized urinary bladder matrix (UBM) is treated as superior and inferior surface layers. The interlayer is totally encapsulated by the mentioned superior and inferior surface layers, forming a sandwich structure with advantages of integrating UBM and SIS to have high bioactivity with bionic structure, UBM isolates the immunogenicity of SIS and direct contact with host tissue, and after implantation the basic type of inflammatory interaction in the host-implant marginal zone is the same as that of pure UBM, with high biocompatibility; effective endotoxin removal optimize the biosafety of the material after implantation; feasibility for industrial large-scale production; the stiffness of the material can be maintained even after hydration, with good handling feel and fit condition, beneficial for the suture fixation and also shorten the fixation or surgery time.

### TECHNICAL FIELD

The present invention relates to the field of materials for tissue repair, in particular, a biological material with composite extracellular matrix component

### BACKGROUND OF THE INVENTION

Acellular tissue matrix (ACTM) is an important progress made in the study on the material for soft tissue repair in recent two decades. Cells, antigens, lipids, soluble proteins, and the like in raw tissues are removed completely by physical or chemical methods, and the resulted insoluble extracellular matrix (ECM) with highly preserved appearance, histological characteristics, and ultrastructure is used as biological scaffold, namely ACTM. ECM is highly conserved in the process of biological evolution, and ECM of the same tissues slightly differs among different species. Therefore, ACTM deprived of cell components and antigens that may cause immunological rejections by decellularization process can be used safely as allografts and xenografts. When used to repair tissue defects, synthetic material induces chronic inflammatory stimulations that recruit fibroblasts to form scars; differing from the aforesaid mechanism, ACTM can induce endogenous tissue regeneration, i.e., biological signals and degradation products from the implanted ACTM can induce macrophages and stem cells locates around the region under repair to infiltrate scaffold, to grow and proliferate rapidly, and to secrete their own extracellular matrix to substitute implants. Host tissue grows in ACTM while the later degrades gradually, these two processes are simultaneous basically, and in the end ACTM will be replaced completely by host tissue. Therefore ACTM as the material for tissue repair manifests more advantages: \textcircled{1} Its structure and components are close to those of natural tissues: its main components are collagen fibers and other structural proteins, with trace glycoprotein, fibronectin, glycosaminoglycan and proteoglycan, growth factors, enzymes, etc., \textcircled{2} ACTM shows reasonable and controllable degradability, which accords better with the pattern of regeneration; \textcircled{3} ACTM possesses reasonable porosity, which benefits the interchange of substances and air among tissues; \textcircled{4} ACTM possesses reasonable mechanical strength, so that it can support the ingrowth of tissues; \textcircled{5} ACTM possesses tolerance to infection, and therefore it can be used to repair contaminated or potentially contaminated tissue defects, which may be attributed to timely ingrowth of phagocytes, early and local revascularization, so that bacterial biofilm is hard to form.

At present, ACTM is clinically used as substitutes for meninx, pleura, abdominal wall, fascia, etc., as well as used in stoma reinforcement, pelvic fundus reconstruction, urinary bladder suspend, injured solid viscera (such as spleen, liver) packing hemostasis, and treatments of various complex hernias and abdominal wall defects, such as contaminated abdominal wall defects, infection after implantation of synthetic materials, reoperation of intestinal fistula, etc. Statistically, ACTM accounted for 5˜10% of all materials used in the repair of soft tissue defects in US. Biological mesh can be divided into following two main types depending on the source of raw materials: \textcircled{1} Materials from inert tissue (IT), represented by human/porcine dermis, bovine/porcine/equine pericardium, and bovine/porcine peritoneum. These materials are derived from biologically inert tissues in the body, consisted of almost only structural proteins (comprises collagen fibers and elastic fibers), and absence of bioactive components such as fibronectin, growth factors, and proteoglycan. \textcircled{2} Biological materials from ECM, possess intact 3D-ultrastructure of extracellular matrix from live tissues and bioactive components such as fibronectin, growth factors, and glycosaminoglycan, represented by small intestinal submucosa (SIS), human amniotic membrane, and urinary bladder matrix (UBM). The technology for preparing biological materials of ECM sourced (including decellularization and shaping) is more complex than that of IT tissue source, however, the former is superior to the later in terms of the repair efficacy of tissue defects: \textcircled{1} The implantation of IT sourced materials can only induce the regeneration of vascularized connective tissues to fill in and anatomically repair the defects, while ECM biological materials can integrate effectively with host tissue, actively induce autologous stem cells to migrate to the injured site, and promote their proliferation and differentiation to realize tissue-specific and functional repair to some degrees; for example, ECM biological materials can be used to realize the regeneration of muscle and fascia, partial recovery of innervation to improve the function of disabled extremities, and reconstruction of fingertips, etc. \textcircled{2} Materials of IT source are dense in structure and contain a lot of elastic fibers that degrade slowly and cannot autologous renew after the age of 25, leading to long-term instability and loss of elasticity in implanted regions. \textcircled{3} In contrast with biological materials of IT source, those of ECM source possess better tolerance to infection and faster tissue regeneration.

SIS is obtained from mammalian small intestine that is delaminated mechanically to remove serosa, mucosa and muscularis layers and then decellularized. SIS is mainly consisted of type-I and type-III collagen, and trace type-IV and type-V collagen, glycosaminoglycan, growth factor, fibronectin, etc. Implanted SIS can degrade completely, and therefore it is a favorable scaffold in tissue engineering. SIS possesses good mechanical strength, broad source of raw materials, and easier or automated pre-treatment. However, its bioactivity is relatively low, and it carries higher original bioburdens because its raw material may contact various antigens in food in vivo, therefore these bioburdens (likely endotoxin, Gal epitope residue) reserve immunogenicity even after harsh decellularization and sterilization, which causes host responses.

China Patent No. CN2608014 disclosures an artificial dura mater, which is prepared by bonding small intestine submucosa to human amniotic membrane in order that it can have the mechanical tensile strength of human dura mater and anti-adhesion ability of arachnoid membrane both. However, the aforesaid method of preparation dose not completely isolate the immunogenicity of SIS.

China Patent No. CN101366979 disclosures a tissue patch and related methods of preparation; in the said tissue patch, decellularized small intestine submucosa is used as internal layer that is encapsulated with decellularized amniotic membrane. Decellularized amniotic membranes isolated the immunogenicity of decellularized small intestine submucosa, and decellularized small intestine submucosa reinforced the mechanical strength of decellularized amniotic membranes. The said tissue patch shows higher bioactivity and histocompatibility, meantime no significant immunological rejection or cytotoxicity. However, the amniotic membrane is a material of human origin, so that its source is difficult to control; in addition, it carries the risk of transmitting unknown viruses or diseases. Furthermore, small intestine submucosa is a material of porcine origin. Raw materials of multiple sources require complex tracing systems, and therefore large-scale production of the said tissue patch is infeasible.

The main component of UBM is basement membrane (BM). In contrast with SIS, UBM shows following advantages:

(1) Very low immunogenicity and high histocompatibility: UBM is a kind of highly pure extracellular matrix material, which is free from endotoxin, bioburden or cell debris. UBM do not contact the bioburden before harvest such as bacteria in vivo with simple hierarchy structure, so that endotoxin contamination can be avoided in the process of harvest. Pure UBM is capable of being implanted within a human or animal patient without causing cytotoxic response, infection, rejection of the implant or any other harmful effect to the patient.

(2) High bioactivity: UBM contains intact structure and components of basement membrane Degradation products of UBM contain over 5000 kinds of active components, of which 41 kinds of proteins or polypeptides have been proven to be associated with wound healing, their functions including nourishing nerves, promoting revascularization, and inhibiting tumor growth, etc.

BM, not submucosa, is the main factor in supporting the metabolism of tissues and organs: \textcircled{1} take small intestine as an example, it is used to believe that SIS supports the 3-day update of small intestinal mucosal cells, thus SIS is widely utilized as raw material of repair material at present; the inventor discovered that there is BM contents located on the surface of SIS, which supports the metabolism of mucosal cells. After the removal of BM contents on the SIS, laminin, fibronectin, glycosaminoglycans, growth factors and other bio-active contents of the residual material is undetectable; \textcircled{2} All organs and tissues in the body contain basement membrane+connective tissue structure (the submucosa is a connective tissue), and the key to self-tissue repair/remodel lies in the integrity of the basement membrane layer. For example, the periodic proliferation and shedding of the endometrium depends on the integrity of the endometrial basement membrane. Once the basement membrane is damaged, it will cause functional disorders. For patients who used Matristem (UBM product manufactured by Acell Inc.), the healing time for open wounds was shortened from 25.5 weeks to 9.8 weeks (J Wound Care 2012; 21:476, 478-480, 482). In the process of production, however, the delamination of UBM costs a lot of labor; in addition, it is difficult to fabricate UBM into products of proper thickness for its unsatisfied mechanical strength and smooth surface.

(3) BM is highly conserved among different species, highly homologous among different tissues and organs and can be used to repair a variety of tissue defects: \textcircled{1} ECM is highly different among different species, and BM is the most conservative part of ECM. \textcircled{2} the inventor carried out a series of original scientific researches and experiments concerning BM derived from different tissues and organs (BM on the surface of small intestine SIS, bladder BM, endometrial BM, aortic BM, skin BM, etc.) such as: scanning electron microscopy, histological staining (HE staining, Masson's trichrome staining) analysis of ultrastructure; specific staining (Movat's staining, Van Gieson staining, etc.), immunohistochemical staining (vimentin, desmin, smooth muscle actin, keratin, vascular endothelial factor, etc.), cytometric bead array by flow cytometer (growth factors, cytokines, etc.) to analyze active components; isotope labeling to observe in vivo metabolic pathways; mass spectrometry separation and proteomics analysis to observe the components and functions of metabolic degradation products by protease and acidolysis treatments; comparison of cell proliferation and migration of different source cells (small intestinal mucosal cells), urinary epithelial cells, endometrial cells, vascular endothelial cells, dermal fibroblasts) on different sources of BM, and finally confirmed that BM is highly homologous among different tissues and organs, that is, theoretically, a tissue organ BM (such as UBM) can repair other organ and tissue defects.

(3)

In the clinical application of biological materials, it is inevitable that they will come in contact with blood or body fluids and subsequently undergo hydration. However, hydrated biomaterials have the problems of being soft, hard to fit in, suture difficulties and fixing troubles after hydration, which increase the operation difficulties for surgeons and also prolong the operation time. For membrane-like products that need to be sutured and fixed when implanted into human body, it is difficult to stretch and suture too soft materials. However, too stiff material cannot fit the wounds to preform proper sutures. Therefore, adjusting the stiffness of the material has clinical significance. Chinese invention patent CN107335097A attempts to add synthetic fibers between the material layers to improve the mechanical strength and flexibility of the material after hydration, but the degradation products of degradable synthetic fibers are acidic substances, which are not conducive to the healing of tissue defects; while non-degradable synthetic fibers may cause material shrinkage, chronic erosion, pain and other problems.

In addition, endotoxin is one of the important causes of inflammation-related complications in the implanted materials. FDA has set endotoxin limits for the implanted medical devices to below 0.5 EU/mL or 20 EU/device. However, for collagen-based materials, endotoxin could easily adhere to the triple-helix structure of collagen fibers and be hidden in the structure. Once exposed to endotoxin contamination during raw material harvest and preliminary processing, it is difficult to extract endotoxin using conventional decellularization reagents from the collagen matrix. In addition, endotoxin has a masking effect. Masked endotoxin cannot be completely detected by limulus amebocyte lysate (LAL)-based assays, but it can trigger systemic inflammatory responses even at very low concentrations, which can have an adverse effect on the biological safety of the implanted materials.

### SUMMARY OF THE INVENTION

The present invention aims to solve a technical problem as to how to provide a biological material with composite extracellular matrix component that integrates merits of UBM and SIS both: \textcircled{1} High bioactivity with bionic structure, the present invention helps to realize specific and functional repair to some degrees with only slight tissue adhesion and without excessive scars; \textcircled{2} UBM isolates the immunogenicity of SIS and the direct contact of SIS with host tissues; after the implantation of the said biological material, the basic inflammatory response in the host-implant marginal zone is the same as that of pure UBM, with high biocompatibility; \textcircled{3} SIS can make up the disadvantage of UBM—low mechanical strength; furthermore, the preparation of SIS is easier, and its thickness can be changed by composition; \textcircled{4} the present invention with raw materials of same origin are feasible for industrial large-scale production; \textcircled{5} the stiffness of the material can be maintained for a long time even after hydration, with good handling feel and fit condition, which is beneficial for the suture fixation and also shorten the fixation or surgery time. Therefore, the said biological material can be used in the filling, reinforcement, repair or reconstruction of fascia, meninx, pleura, pelvic fundus, derma, solid viscera and various soft tissue defects, possessing good clinical practicability.

The present invention relates to a biological material with composite extracellular matrix components, wherein decellularized small intestinal submucosa (SIS) is used as interlayer of the biological material, decellularized urinary bladder matrix (UBM) is used as a superior surface layer and the inferior surface layer of the biological material, and the superior surface layer and the inferior surface layer encapsulated completely the interlayer to form a sandwich structure.

The thickness of the superior surface layer is 0.05 mm-0.2 mm.

The thickness of the inferior surface layer is 0.05 mm-0.2 mm.

The bending length of the biological material was reduced by no more than 50% after 10 min of hydration.

After the biological material is hydrated, the bending length should not be reduced by more than 50%.

After the biological material is hydrated, the bending length should not be reduced by more than 50% without changing the overall thickness of the material. Moreover, in clinical application, fixation time and operation time of the biological material of the present invention are significantly shortened compared with pure SIS material.

UBM and SIS were separately processed for endotoxin removal; the endotoxin removal process is as follows: the UBM and SIS is treated with lipid reduction process to reduce the lipid content to less than 2% and subsequently treated with an alkali solution. Specifically, the endotoxin removal process can be comprised in the decellularization processing, and the process may be before or after any step in the decellularization process.

Specifically, the lipid reduction process is organic solvent treatment for 2-16 h;

Preferably, the organic solvent treatment is as follows: immense and vibrate the materials in the solvent with several solvent changes.

Preferably, the materials should be dried before the organic solvent treatment, the water content of the dried materials should be less than 10%.

In the alternative, organic solvent comprises ethanol, methanol, chloroform and other organic solvent that can be used to remove lipids from the materials.

In the alternative, lipase, detergent and organic solvent can be used to reduce the lipid content in the biological materials.

Preferably, the lipid content should be reduced to less than 1% followed by an alkali solution treatment.

Specifically, alkali solution treatment is 0.1-2% (w/v) alkali solution treatment for 0.25-1 h.

Preferably, the alkali comprises sodium hydroxide, potassium hydroxide.

The endotoxin content of the biological material can be reduced to less than 0.5 EU/g, preferably, the endotoxin content of the biological material is less than 0.1 EU/g.

Lipid reduction and alkali treatment should be successive steps. Lipid reduction treatment of the endotoxin removal process is to effectively remove lipids, so that the cell membrane is destroyed, the hydrophilicity and the permeability of the material is increased, and most of the bioburden can be removed; and facilitating the subsequent alkaline solution treatment to achieve a thorough endotoxin removal quickly. The applicant discovered that a lipid residue of less than 2%, preferably less than 1%, is suitable for the subsequent alkali treatment, and can maximize the effectiveness of endotoxin removal. Although the lipid reduction or alkali treatments have been used separately in a decellularization treatment, the present invention can achieve the purpose of completely removing endotoxins by adjusting the process parameters and applying the two continuously, without extending the production cycle, and ensuring the production efficient.

The SIS is obtained from mammalian small intestine that is delaminated mechanically to remove serosa, mucosa and muscularis layers and then decellularized, and is a membrane-like material.

The UBM is obtained from mammalian urinary bladder that is delaminated mechanically to remove serosa, muscularis external, submucosa and muscularis mucosa layers and then decellularized, and is a membrane-like material.

A number of layers in the said interlayer is 1˜20.

A number of layers in the said superior or inferior surface layers is 1˜10 respectively.

A interlayer is bonded between the superior surface layer and the inferior surface layer by one or several of such methods as medical adhesive, suturing and tying, and vacuum pressing; layers in interlayer, superior and inferior surface layers (among multilayered UBM and multilayered SIS) are bonded among them with the aforesaid method too.

The superior surface layer and the inferior surface layer possess high bioactivity, and they can isolate effectively the immunogenicity of interlayer and do not change the type of inflammatory responses of pure UBM in the marginal zone of host tissues. The interlayer can improve remarkably the mechanical strength and thickness of the said biological material.

The medical adhesive is one or several of chitosan, collagen, fibrin glue, hyaluronic acid, chondroitin sulfate, hydrogel, bone glue, gelatin, or pectin. Preferably, absorbable medical adhesives and sutures.

A technical parameter of the said vacuum pressing is Vacuum pressure: −50˜−760 mmHg, acting duration: 0.5˜72 h.

The biological material comprises also perforations that penetrate the biological material.

A diameter of the perforations is 1˜5 mm, and a spacing between the perforations is 0.5˜5 cm.

The biological material is applied to laparoscopic repair of inguinal hernia, femoral hernia, and abdominal wall hernia.

### Effects of the Invention

(1) High bioactivity, with bionic structure, the present invention helps to realize specific and functional repair to some degrees with slight tissue adhesion and no excessive scars; intact basement membrane is the indispensable basis for functional complete self-repair in vivo: \textcircled{1} intact basement membrane components of the surface layer can release a lot of active factors to support and regulate such live activities as growth and differentiation of cells; for example, released basic fibroblast growth factor, epithelial growth factor, hepatocyte growth factor, and keratin growth factor to promote cell adhesion and migration, induce cell differentiation, and reduce cell apoptosis, which can make up for the shortage of regenerative activity in SIS; \textcircled{2} “submucosa+basement membrane” as a bionic structure; all the tissues and organs contain the structure of basement membrane+connective tissue (submucosa is a kind of connective tissues), in the present invention, SIS is beneficial for the growth of mesenchymal cells, and UBM for the adsorption and proliferation of epithelial cells which are very helpful for the wound healing of skin, blood vessels, mucosa, and epithelium; \textcircled{3} it regulates the infiltration of epithelial cells to precede that of fibroblasts, inhibits the secretion of excessive fibrinogen, thus epithelial tissue will be formed before fibroblasts predominate, and the smooth surface of basement membrane of the said biological material helps to reduce adhesion and inhibit the formation of scar tissue.

(2) High histocompatibility, low immunogenicity, and same basic type of inflammatory response as pure UBM in the host-implant marginal zone: SIS is encapsulated by UBM with very low immunogenicity, so that SIS cannot directly contact host tissue in the early period of implantation to retard the release of components that may cause immune responses, and the type and severity of immune responses of the said biological material would be the same as those of pure UBM. The degradation of UBM and tissues ingrowth are simultaneous, and the gradual exposure of SIS in the late period of implantation would not change the results of tissue repair. Endotoxin residue in the material is effectively removed, with decellularization process, resulting in decreased incidence of inflammation-related complications

(3) The composite material with an UBM surface layer of a thickness of 0.05-0.2 mm can maintain the stiffness of the material for a long time after hydration, with good handling feel and fit condition, which is beneficial for the suture fixation and also shorten the fixation or surgery time. By changing the thickness of the surface layer, the stiffness of the material after hydration can be further adjusted without increasing the overall thickness of the material.

(4) Decreased difficulty in the pretreatment of raw materials and lower price of the product: While combining merits of both UBM and SIS, the present invention can greatly reduce the usage of UBM, which is a limiting factor to yield and cycle of production, thus the cost of raw materials can be lowered, the cycle of production shortened, and the input of human labor reduced.

(5) the composite material is remodelable, can be applied to the laparoscopic repair of inguinal hernia, femoral hernia, and abdominal wall hernia.

Laparoscopic repair with the advantages of minimal incision, less tissue trauma and faster recovery for patients has been widely used in the repair of inguinal hernia and abdominal wall hernia. The materials used for hernia repair can be classified as bio-remodelable materials and synthetic materials or non-degradable materials. Compared with synthetic materials, bio-remodelable materials possess advantages such as complete degradation, no permanent foreign body remaining, no chronic pain, no delayed infection or other complications that related to the synthetic materials, which is ideal for hernia repair. The tissue repair mechanism of bio-remodelable materials is endogenous tissue regeneration, its repair efficacy depends on the ability to induce neo-tissue formation. However, in laparoscopic hernia repair, hernia ring is not closed and the materials is being placed at the hernia defects with a lack of blood supply, abundant collagenase and high mechanical properties requirements, posing great challenges for tissue regeneration. IF the implanted material is degraded before that neo-tissue with sufficient strength is formed, a recurrence might occur. At present, no commercially available bio-remodelable material can be used in laparoscopic hernia repair. Only synthetic materials, which can form a stiff scar-like tissue in the repair area, can be used in laparoscopic hernia repair.

The present invention provides a bio-remodelable material that can be applied to laparoscopic hernia repair. Its efficacy has been confirmed by randomized clinical trial to be accurate and reliable, and fills in the gap of no bio-remodelable material applicable for laparoscopic hernia repair. In addition, the incidence of complications can be reduced and the delayed infection, organ erosion, chronic pain and other complications that may exist in the synthetic material repaired patients can be fundamentally avoided.

### BRIEF DESCRIPTION OF THE DRAWINGS

FIG. 1 shows the structure of the present invention, wherein digit 1 indicates decellularized small intestinal submucosa (SIS), and digit 2 indicates decellularized urinary bladder matrix (UBM)

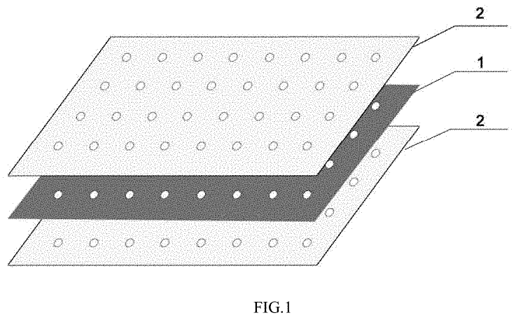

### DETAILED DESCRIPTION OF THE PREFERRED EMBODIMENT

Following specific embodiments are used to further expound the present invention. Those embodiments are only for explaining the present invention, and shall not limit the scope of the present invention. It shall be understood that technicians in the same field can make various changes or modifications after they read the content of the present invention, and all those changes and modifications are equivalent to the present invention and therefore fall within the scope of the present invention.

### Example 1

Porcine decellularized urinary bladder matrix (UBM) and decellularized small intestinal submucosa (SIS) were prepared. Spread a monolayered UBM out smoothly (the smooth surface being downward), composite SIS into an independent layer, each SIS being overlapped by 50%. Then place 4 aforesaid independent layers on the aforesaid UBM, each layer being interlaced by 90°. Place a monolayered UBM on the surface of the aforesaid layers (the smooth surface being upward). Dissipate bubbles, bind all interlayers with medical chitosan as adhesive, then press the above layers under −250 mm Hg for 24 h to make it become a whole material. The aforesaid material is perforated through all layers, the hole spacing being 5 mm, and the diameter of hole being 1 mm.

### Example 2

Porcine decellularized urinary bladder matrix (UBM) and decellularized small intestinal submucosa (SIS) were prepared. Spread 2 layers of UBM out smoothly (the smooth surface being downward), composite SIS into an independent layer, each SIS being overlapped by 50%, then place 6 aforesaid independent layers on the surface of UBM, each layers being interlaced by 90°. Spread 2 layers of UBM on the surface of the aforesaid layers (the smooth surface being upward). Dissipate bubbles, bind all interlayers with medical collagen as adhesive, then press the above layers under −300 mm Hg for 36 h to make it become a whole material. The aforesaid material is perforated through all layers, the hole spacing being 8 mm, and the diameter of hole being 2 mm.

### Example 3

According to GB/T528-2009, 3 samples were taken, each being 4 cm×1 cm in size and dumbbell-like in shape; aforesaid 3 samples were hydrated and then their two ends were fixed to a mechanical tester and pulled at the speed of 10 mm/min, and the tensile strength of those samples was 34±3 N/cm.

Three samples were taken and cut into 2 cm×5 cm in size; two ends of those samples were fixed to upper and lower clips of a tensile machine respectively, and those samples were peeled continuously at the speed of 10 mm/min till the overlapped part of them laminated; the force at stratification was recorded. The peeling strength of SIS-SIS and UBM-SIS was 6±2 N/cm, and the force for maintaining peeling was 1.5±0.5 N/cm.

The cytotoxicity of the said material was evaluated by the method claimed in GB/T 16886.5. NIH3T3 cells and L929 cells were used, and cell culture medium was used as extracting agent, extracts of gradient concentrations were used as cell culture media, and MTT method was used to determine the cell viability. The cytotoxicity of the said biological material was graded to be 0˜1.

Cell migration: The said material was powered under low temperature and then degraded by protease, the concentration of enzymatic products being 50 μg/mL. Cells were starved for 24 h, and Boyden chamber method was used to determine 6 h-migration of cells, medium with and without 10% fetal bovine serum being used as positive and negative control respectively. Migrated cells for the said material was 2056±72, and that for positive control was 2105±35, that for negative control was 1328±65. No significant difference was detected between the said material and positive control regarding cell migration (P>0.05).

The hemocompatibility of the said material was determined by the method claimed in GB/T14233.2. Contact group: The back of rats was dehaired, applied with 50 μg/mL enzymatic products for once a day, consecutive 20 days. Oral administration group: 1 ml of extract was administered orally every other day within 7 days, 4 administrations in total; intramuscular injection and intravenous injection group: 0.15 mL of extract was injected every other day within 7 days, 4 injections in total. Rats in aforesaid 4 groups were killed 30 days and 90 days respectively after the administration, and the venous blood was collected for detection. The hemolytic ratio was calculated by the following formula: Hemolytic rate (%)=(absorbance of sample minus absorbance of negative control) divided by (absorbance of positive control minus absorbance of negative control)×100%. The hemolytic rate of the said material was ≤5%.

The intradermal stimulation of the said material was evaluated by the method claimed in GB/T 16886.10. Rabbits were subject to the intradermal injection of 0.2 mL of extract and 0.2 mL of control (PBS) respectively, and the skin reaction of the injected region was observed 15 min, 1 h, 2 d, and 3 d after the injection; the stimulation grades was scored as erythema and edema. The said material showed no intradermal stimulation.

The sensitization of the said material was evaluated by the maximal dose method claimed in GB/T 16886.10. The solution of pure starch was used as negative control. Extracts of the said material and control were oral administered consecutively for one week. Rats were observed for one week after oral administration. Weight, clinical toxic symptoms and grade of toxicity were daily recorded. All rats were killed after the test, and the pathological examination demonstrated that the said material showed no delayed super-sensitivity.

The animal model with defects of rectus abdominis sheath and rectus abdominis was established in dogs, the defect area being 10×5 cm²; the said material was cut into suitable size for defect repair, pure SIS and pure UBM being used as controls. The incidence of seroma in the repair region was 33% for pure SIS, and no seroma occurred for pure UBM and the said material. Repair region was harvested 2 weeks, 1 month, 2 months, and 4 months respectively after the aforesaid repair and subject to staining of CD68, CCR7, and CD163 to observe the classification and density of infiltrated cells and the ratio of M1 macrophages to M2 macrophages. It was confirmed that the basic type of inflammatory interaction of said material in the host-implant marginal zone was the same as that of pure UBM, and repair efficiency was close to that achieved by pure UBM.

### Example 4

### Bending Length and Flexural Rigidity Measurement:

Control: 8-layer SIS

Sample A: 6-layer SIS+UBM as the upper and lower surface layers, the thickness of surface layer is 0.05 mm

Sample B: 6-layer SIS+UBM as the upper and lower layers, the thickness of the surface layer is 0.1 mm

Sample C: 6-layer SIS+UBM as the upper and lower layers, the thickness of the surface layer is 0.2 mm

Vacuum lamination is used to prepare the sample to be tested.

Spiral thickness meter was used to test the prepared controls and samples, and 5 points for each sample were tested randomly. Average values are shown in Table 1. No significant difference in the overall thickness of materials was observed in each group

An analytical balance was used to test the mass per unit area of the controls and samples. No significant difference was found in the mass per unit area in each group of materials, and the results are shown in Table 1.

TABLE 1
Thickness and mass per unit area of test samples

| | Sample thickness (mm) | Mass per unit area (g/m²) |
|---|---|---|
| Control | 0.25 | 143 |
| Sample A | 0.22 | 132 |
| Sample B | 0.30 | 147 |
| Sample C | 0.25 | 135 |

Methods: The bending length and flexural rigidity of unhydrated materials with a width of 1 cm and a length of 20 cm were measured by the section method. The material was immersed in saline at room temperature for 2 or 10 min After taking the material out, both the bending length and flexural rigidity were measured. Each sample was measured 5 times and the average value was calculated. The results are shown in Table 2. Bending length is defined as the length of a rectangular strip of fabric, fixed at one end and free at the other, that will bend under its own weight to an angle of 7.1°; flexural rigidity is defined as the ratio of the small changes in bending moment per unit width of the material to its corresponding small changes in curvature.

TABLE 2
Bending length and flexural rigidity

| | Bending length | Bending length | Bending length | flexural rigidity | flexural rigidity | flexural rigidity |
|---|---|---|---|---|---|---|
| | (before | (2 min after | (10 min after | (before | (2 min after | (10 min after |
| | hydration, cm) | hydration, cm) | hydration, cm) | hydration, mN · cm) | hydration, mN · cm) | hydration, cm) |
| Control | 6.5 | 1.3 | 1.0 | 39.3 | 0.7 | 0.4 |
| Sample A | 6.2 | 4.0 | 3.4 | 31.5 | 16.9 | 11.4 |
| Sample B | 6 | 4.5 | 3.7 | 31.3 | 26.8 | 15.8 |
| Sample C | 6 | 4.8 | 4.2 | 29.2 | 27.9 | 21.0 |

According to Table 2, the bending length of the samples A, B and C did not exceed 50% of the values, when “10 min after hydration sample” was compared to “before hydration sample”. Thus, the structure of UBM as the upper and lower layers and SIS as the middle layer and the thickness of UBM is in the range of 0.05-0.2 mm, can reduce the bending length of the whole material by no more than 50%, after hydration for 10 min. When the thickness of the overall material remains unchanged, as the thickness of the UBM increases, the reduction of the bending length after hydration become less, leading to better operation performance.

Clinical research summary: A randomized, single-blind, parallel-controlled, multi-center clinical trial was used to evaluate the effectiveness and safety of the biological material for the treatment of inguinal hernia. There are 150 patients of open surgery, including 75 cases in the control group and 75 cases in the experimental group. The test device was sample A shown in the example 4, and the control device was the control shown in example 4 (porcine SIS products). The main observation index was the recurrence rate of inguinal hernia at 6 months after operation. The secondary safety evaluation indexes were postoperative fever, hematoma/seroma in the operation area, evaluation of infection in the incision area, pain scale, foreign body sensation in the groin area, and allergic reactions.

Results:

Recurrence rates of hernia at 6 months after operation

Experimental group: None.

Control group: 2 cases.

Recurrence rates after 18 months

Experimental group: None.

Control group: 4 cases.

Other indexes:

The material operation feeling of the experimental group was better than that of control group. The material fixation time in the experimental group was shorter than the control group, making the operation time significantly shorter than the control group.

TABLE 3
Patch operation time

| Item | Control group | Experimental group |
|---|---|---|
| Patch fixation time | 12.02 ± 4.83 | 9.89 ± 4.12 |
| Operation duration | 68.06 ± 18.10 | 63.06 ± 18.00 |
| (min) | | |

Based on the above clinical results, the biomaterial of the present invention has a good operating feel. The biomaterial can maintain the stiffness of the material after hydration and had a good fit, which were conducive to surgical suture operations and shorten the material fixation time.

### Example 5

Porcine SIS was the experimental material. SIS was separated from the freshly obtained porcine small intestine, and it was cleaned briefly with peracetic acid and phosphate buffer, disinfected, freeze-dried and used as sample 1.

After Sample 1 was cleaned with a mixture of chloroform/ethanol (3:1, v/v) for 10 times as degreasing treatment, the lipid content was detected to be 1.2%, which was used as sample 2. Next, sample 2 was rehydrated step by step to remove organic solvent residues, treated 0.2% sodium hydroxide solution for 60 min, rinsed with injection water, freeze-dried and then used as sample 3.

Sample 1 was treated 0.2% sodium hydroxide solution for 60 min, rinsed with injection water, freeze-dried and used as sample 4.

For preparation of sample 5, methods described in U.S. Pat. No. 6,206,931 were used. Adult porcine small intestine was washed with water and treated with 0.2% peracetic acid/5% ethanol aqueous solution for 2 h. SIS was separated and freeze-dried.

The samples were layered and made into materials for the tests. Each material was cut into small pieces to detect the endotoxin content: Endotoxin-free water was used for 2 h extraction procedure. Dynamic spectrophotometry was used to detect the endotoxin content of the material. Whenever needed, a gradient concentration dilution was done before measuring the endotoxin content. The test results are shown in Table 4:

TABLE 4
The endotoxin content of the samples

| | Endotoxin content (EU/g) |
|---|---|
| Sample 1 | 786.98 |
| Sample 2 | 4.98 |
| Sample 3 | 0.05 |
| Sample 4 | 467.32 |
| Sample 5 | 1.37 |

The endotoxin detection has the problem of false negative or false positive result. So, in order to further clarify the endotoxin removal effect, macrophage activation method was used as a semi quantitative analysis to detect the activity of endotoxin residue. If the secretion of TNF-α is higher, more severe inflammatory reactions are expected after implantation, which reflects a higher endotoxin content present in the implant.

THP-1 mononuclear macrophages in the logarithmic phase were seeded into a 24-well culture plate at a density of 1×10⁴ cells/well, and cultured overnight at 37° C. After overnight culture, the cells were replenished with fresh media. The samples from each group were cut into 1×1 cm² and placed in the upper chamber of the transwell, which was placed in the 24-well culture plate with an addition of 0.5 mL culture medium to the upper chamber. After co-culturing for 24 h, the cell culture supernatant was collected, centrifuged and used for the detection of TNF-α level by ELISA. Alamar blue reagent was added to the cells for staining and counting the live cells. LPS was used as the positive control and the complete medium served as the negative control. The TNF-α content of each test sample was shown in Table 5.

The results in Table 5 showed that the method of degreasing and alkali treatment, as disclosed in the present invention, is effective for endotoxin removal. This method thoroughly removes endotoxins from raw materials.

TABLE 5
TNF-α content in the samples

| | TNF-α content (pg/50000 cells) |
|---|---|
| Sample 1 | 674.2 |
| Sample 2 | 217.6 |
| Sample 3 | 4.9 |
| Sample 4 | 441.3 |
| Sample 5 | 95.4 |
| Positive control | 2,049.5 |
| Negative control | 2.3 |

### Example 6

Porcine SIS was the experimental material. SIS was separated from the freshly obtained porcine small intestine, and it was cleaned briefly with peracetic acid and phosphate buffer, disinfected, freeze-dried and used as sample 6.

After Sample 1 was cleaned with lipase for 12 h at 4° C., and then freeze dried, followed by treatment of chloroform/ethanol (1:1, v/v) for 2 times. The lipid content was detected to be 1.2%, which was used as sample 7. Next, sample 7 was rehydrated step by step to remove organic solvent residues, treated 1.5% sodium hydroxide solution for 60 min, rinsed with injection water, freeze-dried and then used as sample 8.

Sample 6 was treated 1.5% sodium hydroxide solution for 60 min, rinsed with injection water, freeze-dried and used as sample 9.

The samples were layered and made into materials for the tests. Each material was cut into small pieces to detect the endotoxin content: Endotoxin-free water was used for 2 h extraction procedure. Dynamic spectrophotometry was used to detect the endotoxin content of the material. Whenever needed, a gradient concentration dilution was done before measuring the endotoxin content. The test results are shown in Table 6:

TABLE 6
The endotoxin content of the samples

| | Endotoxin content (EU/g) |
|---|---|
| Sample 6 | 786.98 |
| Sample 7 | 5.30 |
| Sample 8 | 0.38 |
| Sample 9 | 345.61 |

1. A biological material with composite extracellular matrix components, comprising:
an interlayer containing a decellularized small intestinal submucosa (SIS);
a superior surface layer containing a decellularized urinary bladder matrix (UBM);
an inferior surface layer containing a UBM;
wherein the superior surface layer and the inferior surface layer encapsulated completely the interlayer to form a sandwich structure;
wherein the thickness of the superior surface layer is 0.05 mm-0.2 mm;
wherein the thickness of the inferior surface layer is 0.05 mm-0.2 mm;
wherein the SIS were processed for effective endotoxin removal;
wherein the effective endotoxin removal process is as follows: the SIS is treated with lipid reduction process to reduce the lipid content to less than 2% and subsequently treated with an alkali solution.

2. A biological material with composite extracellular matrix components of claim 1, wherein the bending length of the biological material is reduced by no more than 50% after 10 min of hydration.

3. A biological material with composite extracellular matrix components of claim 1, wherein the endotoxin content of the biological material is less than 0.5 EU/g;
preferably, the endotoxin content of the biological material is less than 0.1 EU/g.

4. A biological material with composite extracellular matrix components of claim 1, wherein the lipid reduction process comprises organic solvent treatment for 2-16 h.

5. A biological material with composite extracellular matrix components of claim 1, wherein alkali solution process comprises 0.1-2% (w/v) alkali solution treatment for 0.25-1 h, wherein said alkali comprises sodium hydroxide, potassium hydroxide.

6. A biological material with composite extracellular matrix components of claim 1, wherein the SIS is treated with lipid reduction process to reduce the lipid content to less than 1%, and followed by alkali treatment, and then detergent treatment.

7. A biological material with composite extracellular matrix components of claim 1, wherein the number of layers in the interlayer is 1˜20; wherein the number of layers in the superior surface layer and the inferior surface layer is 1˜10 respectively.

8. A biological material with composite extracellular matrix components of claim 4, wherein the said interlayer is bonded to the superior surface layer and the inferior surface layer by one or several of such methods as medical adhesive, suturing and tying, and vacuum pressing;
wherein the layers in interlayer, superior surface layer and inferior surface layer are bonded among them with the aforesaid method too.

9. A biological material with composite extracellular matrix components of claim 6, wherein the technical parameters of the vacuum pressing are as follows: Vacuum pressure: −50˜−760 mmHg, acting duration: 0.5˜72 h.

10. A biological material with composite extracellular matrix components of claim 1, wherein the biological material is applied to the laparoscopic repair of inguinal hernia, femoral hernia, and abdominal wall hernia.
